(19) United States
(12) Patent Application Publication
(10) Pub. No.: US 2007/0066837 A1
(43) Pub. Date: Mar. 22, 2007

(54) Podophyllotoxin derivatives as antitumor agents

(76) Inventors: Ahmed Kamal, Hyderabad (IN); Mohammed Arifuddin, Hyderabad (IN); Banala Ashwani Kumar, Dist. Nalgonda (IN); Sunanda Ghose Dastidar, New Delhi (IN)

Correspondence Address: RANBAXY INC., 600 COLLEGE ROAD EAST, SUITE 2100, PRINCETON, NJ, 08540, US

(21) Appl. No.: 10/545,838

(22) PCT Filed: Feb. 13, 2004
(86) PCT No.: PCT/IB04/00376

§ 371 Date: Dec. 1, 2006

(30) Foreign Application Priority Data
Feb. 18, 2003 (IN) 139/DEL/2003

(51) Int. Cl. C07D 493/02

(52) U.S. Cl. 549/298; 544/149; 546/283.7

(57) ABSTRACT

This invention relates to podophyllotoxin derivatives, more particularly to 4β-amino and 4β-amido derivatives of podophyllotoxin and 4′-O-demethylepipodophyllotoxin, which are useful for the treatment of tumors. Processes for the preparation of the compounds disclosed herein, pharmaceutical compositions containing these compounds, and methods for treating tumors are provided. The invention further relates to stereoselective compounds of podophyllotoxin and 4′-O-demethylepipodophyllotoxin derivatives.

### FIELD OF THE INVENTION

This invention relates to podophyllotoxin derivatives, more particularly to 4β-amino and 4β-amido derivatives of podophyllotoxin and 4′-O-demethylepipodophyllotoxin, which are useful for the treatment of tumors. Processes for the preparation of the compounds disclosed herein, pharmaceutical compositions containing these compounds, and methods for treating tumors are provided. The invention further relates to stereoselective compounds of podophyllotoxin and 4′-O-demethylepipodophyllotoxin derivatives.

### BACKGROUND OF THE INVENTION

Podophyllum Peltatum, commonly known as the American mandrake or Mayapple, and the related Indian species Podophyllum emodi have been used medicinally for centuries (Catherine et al., Bioor.g Med. Chem. Lett., 22(7), 2897, (1997)). Podophyllotoxin is a bioactive lignan isolated from these plant sources, and this compound has been the focus of extensive chemical modification leading to anticancer drugs, for example etoposide (VP-16) and teniposide (VM-26) (Sakurai et al, Mol. Pharmacol., 40 965, (1991)). These compounds have been used in several human neoplasms, including small cell lung cancer, testicular carcinoma, Hodgkin's disease, Leukemia, Lymphoma, and Kaposi's sarcoma (Jardine, in “Anticancer Agents Based on Natural Products Models”; Academic Press: New York, 319, (1980); Issel, Cancer Chemother. Pharmacol., 7, 73, (1982)). It has been proposed that these and related podophyllotoxins exert their lethal effects by the inhibition of DNA topoisomerase II. These drugs block the catalytic activity of DNA topoisomerase II by stabilizing a cleavable enzyme-DNA complex in which the DNA is cleaved and covalently linked to the enzyme upon treatment with protein denaturants (Osheroff et al., BioEssays, 13, 269, (1991); Alton et al., J. Haematol., 85, 241, (1993)). Topoisomerases are enzymes, which control the topological state of DNA. Type II topoisomerase catalyze DNA strand passage through transient double strand breaks in the DNA. The resulting change in the linking number of DNA allows these enzyme to mediate DNA Interconversions, such as supercoiling and relaxation of super coiling, catenation and de-catenation, knotting and unknotting (Wang et al, Annu. Rev. Biochem., 54, 665, (1985); Maxwell et al, Adv. Protein Chem., 38, 69, (1986)).

Type II DNA topoisomerase enzymes have been shown to be involved in a number of vital cellular processes, including DNA replication and transcription and chromosomal segregation. These enzymes, therefore, are a critical target for the action of a wide variety of anticancer drugs, including etoposide, teneposide. Although, etoposide has been widely used in the clinic, the development of drug resistance, myclosuppression, and poor oral bioavailability has encouraged the medicinal chemists for further synthesis of podophyllotoxin derivatives as useful anticancer drugs.

A number of studies have been carried out on the structural modification of glycoside by amino substituents that are said to have the same or improved inhibitory activity on human DNA topoisomerase II as well as stronger activity in causing cellular protein length DNA breakage (Lee et al, J. Nat. Prod., 52, 606, (1989); Liu et al, Mol. Pharmcol., 36, 8, (1989); Beers et al, J. Med. Chem., 33, 1364, (1990); Zhiyan et al, J. Med. Chem., 45, 2294, (2002); Xiao et al, Pharmaceutical Research, 10(2), (1993); Kuo-Hsiung Lee et al, J. Med. Chem., 33, 1364 (1990); Hong et al, J. Med. Chem., 35(5), 867, (1992); Xiao-Ming Zhou et al, J. Med. Chem., 34, 3346, (1991); Zhe-Qiung Wang et al, J. Med. Chem., 33, 2660, (1990)). Some amino analogs of etoposide have been disclosed in U.S. patent application Ser. No. 07/313826, J. Med. Chem., 33: 1364, (1990); 33: 2660, (1990). WO 93/22319, U.S. Ser. No. 07/987,765, and U.S. Pat. No. 5,300,500.

The amido analogs, which are said to possess anticancer activity, have been disclosed in Japanese Patent No. HI-197486. In the context a large No. of 4β-amido derivatives of podophyllotoxin and 4′-O-demethylepipodophyllotoxin based compounds have been synthesized and investigated for their antitumor activity.

### SUMMARY OF THE INVENTION

The present invention provides 4β-amino and 4β-amido derivatives of podophyllotoxin and 4′-O-demethylepipodohyllotoxin, which are useful for safe treatment of tumors, and method for the synthesis of these compounds. Not only are these compounds more potent than etoposide in the inhibition of human DNA topoisomerase II and in causing protein linked DNA breakage, but these compound also display activity against KB resistant cells.

Pharmaceutical compositions containing the compounds together with pharmaceutically acceptable carrier, excipients or diluents are also provided, which are useful for the treatment of tumors. The pharmaceutically acceptable salts, pharmaceutically acceptable solvates, enantiomer, diastereomers, polymorphs, N-oxides and metabolites of these compounds having the same type of activity are also provided. Pharmaceutical compositions comprising the compounds disclosed herein, their pharmaceutically acceptable acid addition salts, pharmaceutically acceptable solvates, enantiomers, diastereomers, polymorphs, N-oxides or metabolites, in combination with pharmaceutically acceptable carriers and optionally included excipients are also included.

Other aspects and properties of this subject matter will be set forth in the description which follows, and will be apparent from the description or may be learnt by the practice.

In accordance with one aspect, there are provided compounds having the structure of Formula I,

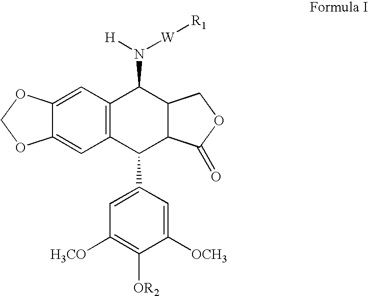

their pharmaceutically acceptable acid addition salts, solvates, enantiomers, diastereomers, metabolites wherein
R₁ can represent alkyl, haloalkyl, aryl, heterocyclic, (CH₂)ₙY (wherein Y can represent halogen, amino, nitro or hydroxyl and n can represent an integer 1 to 4), or (CH₂)ₘZ (wherein Z can represent pyridine, piperidine or morpholine and m can represent an integer 1 to 4).
W can represent no atom, CO, SC, or SO₂.
R₂ can represent hydrogen, or alkyl (C₁-C₃).

In accordance with a second aspect, there is provided a method for treating an animal or human suffering from tumors.

In accordance with a third aspect, there is provided a method for treating an animal or human suffering from tumors, comprising administering to a patient in need thereof, therapeutically effective amount of the compounds as described above.

In accordance with a fourth aspect, there is provided a method for treating an animal or human suffering from tumors, comprising administering to a patient in need thereof, therapeutically effective amount of the pharmaceutical compositions.

In accordance with a fifth aspect, there are provided processes for the preparation of 4β-amino and 4β-amido derivatives of podophyllotoxin and 4′-O-demethylepipodohyllotoxin.

As used herein the term “alkyl” unless otherwise defined refers to a monoradical branched or unbranched saturated hydrocarbon chain having from 1 to 5 carbon atoms. Example of alkyl includes, but are not limited to methyl, ethyl, n-propyl, and the like.

As used herein the term “haloalkyl” refers to alkyl substituted with halogen.

As used herein the term “halogen” refers to fluoro, bromo, chloro or iodo.

As used herein the term “alkoxy” refers to O-alkyl (C₁-C₃).

As used herein the term “thioalkyl” refers to —S-alkyl (C₁-C₃).

As used herein the term “aryl” refers to five or six membered aromatic or fused aromatic radical having 6 to 14 carbon atoms. Examples of aryl include, but are not limited to phenyl, napthyl, anthryl, and the like.

The term “heterocyclic” refers to five or six membered non-aromatic, aromatic or aromatic fused with non-aromatic or aromatic ring system having one or more heteroatom(s) in either the aromatic or the non-aromatic part wherein the said hetero atom(s) can represent nitrogen, sulphur or oxygen, and the ring system includes mono, bi or tricyclic ring. Examples of heterocyclic include, but are not limited to pyridine, pyrimidine, benzothiazole, and the like.

The said aryl and heterocycle may optionally be substituted with one or more substituent(s) independently selected from the group comprising of alkyl (C₁-C₃), haloalkyl (C₁-C₃), alkoxy (C₁-C₃), alkyl (C₁-C₃) amino, thioalkyl (C₁-C₃), halogen, amino, nitro, hydroxy and cyano. The aryl may also optionally be substituted with XA, wherein X can represent CO, CS or SO₂ and A can represent five or six membered aryl or heteroaryl ring optionally substituted with one or more substituent(s) independently selected from the group comprising of alkyl (C₁-C₃), haloalkyl (C₁-C₃), alkoxy (C₁-C₃), alkyl (C₁-C₃) amino, thioalkyl (C₁-C₃), halogen, amino, nitro, hydroxy and cyano.

### DETAILED DESCRIPTION OF THE INVENTION

The compounds described herein may be prepared by techniques well known in the art and familiar to the average synthetic organic chemist. In addition, the compounds of the present invention may be prepared by the following reaction sequences as depicted in Schemes I, II and III.

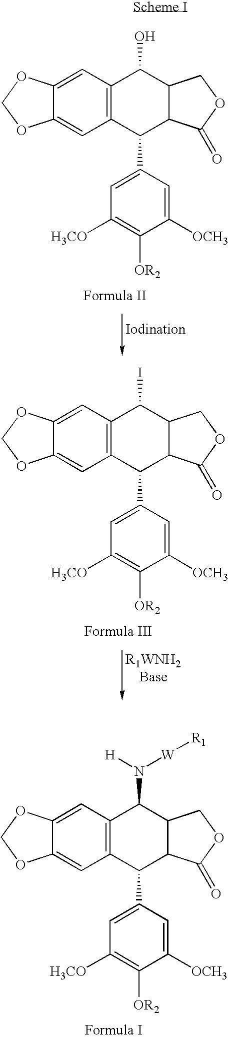

The compound of Formula I can be prepared according to Scheme I. Thus reacting a compound of Formula II with an iodinating agent to give a compound of Formula III (wherein R₂ is the same as defined earlier), which on reaction with a compound of Formula R₁WNH₂ gives a compound of Formula I (wherein R₁ and W are the same as defined earlier).

The reaction of a compound of Formula II to give a compound of Formula III can be carried out in a solvent, for example, methanol, ethanol, tetrahydrofuran, dimethylformamide or acetonitrile. The reaction of a compound of Formula II to give a compound of Formula III can be carried out in the presence of an organic acid, for example, methanesulphonic or p-toluene sulphonic acid.

The reaction of a compound of Formula II to give a compound of Formula III can be carried out in the presence of an iodinating agent, for example, sodium iodide, sodium iodate, potassium dichloroiodate. The reaction of a compound of Formula II to give a compound of Formula III can be carried out at a suitable temperature ranging from 0° C. to 10° C.

The reaction of a compound of Formula III with a compound of Formula R₁WNH₂ to give a compound of Formula I can be carried out in a solvent, for example, tetrahydrofuran, dimethylformamide, methanol, ethanol, dichloromethane or acetonitrile. The reaction of a compound of Formula III with a compound of Formula R₁WNH₂ can be carried out in the presence of an inorganic base, for example, barium carbonate, calcium carbonate, potassium carbonate or sodium bicarbonate.

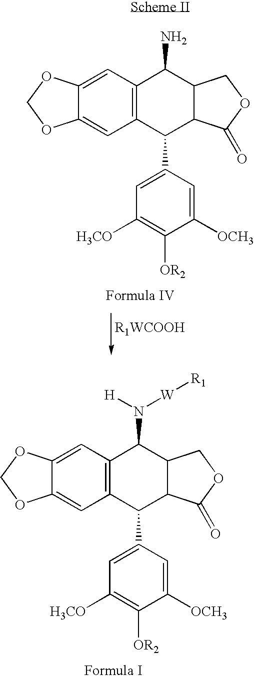

The compound of Formula I can also be prepared according to scheme II. Thus, reacting a compound of Formula IV with a compound of Formula R₁WCOOH to give a compound of Formula I (wherein R₁, R₂ and W are the same as defined earlier). The reaction of a compound of Formula IV with a compound of Formula R₁WCOOH to give a compound of Formula I can be carried out in the presence of an activating agent, for example, dicyclohexyl carbodiimide or 1-ethyl-3(3-dimethylaminopropyl)carbodiimide, in a solvent, for example, dichloromethane, methanol, ethanol, acetonitrile, tetrahydrofuran or dimethylformamide.

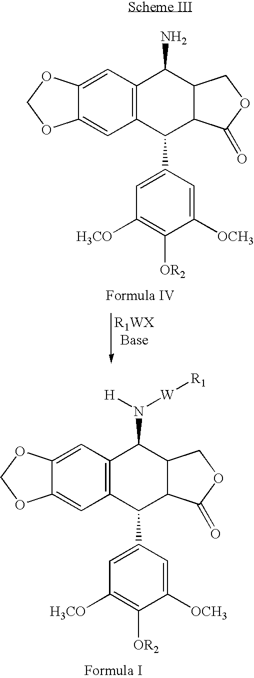

The compound of Formula I can also be prepared according to Scheme III. Thus, reacting a compound of Formula IV with a compound of Formula R₁WX (wherein X is halogen) to give a compound of Formula I (wherein R₁, R₂ and W are the same as defined earlier). The reaction of a compound of Formula IV with a compound of Formula R₁WX to give a compound of Formula I can be carried out in the presence of a base, for example, calcium carbonate, potassium carbonate, triethylamine or pyridine.

Representative compounds capable of being produced by the Schemes I, II and III include
4-β-(4″-Methylbenzophenone-2″-formyl)amino podophyllotoxin (Compound No. 1),
4-β-(3″-Chloro-4″-methylbenzophenone-2″-formyl)amino podophyllotoxin (Compound No. 2),
4-β-(4″-Chlorobenzophenone-2″-formyl)amino podophyllotoxin (Compound No. 3),
4-β-(2″-Chloropyridine-3″-formyl)amino podophyllotoxin (Compound No. 4),
4-β-(6″-Chloropyridine-3″-formyl)amino podophyllotoxin (Compound No. 5),
4-β-(4″-Methylbenzophenone-2″-formyl)amino-4′-O-demethylepipodophyllotoxin (Compound No. 6),
4-β-(3″-Chloro-4″-methylbenzophenone-2″-formyl)amino-4′-O-demethylepipodophyllotoxin (Compound No. 7),
4-β-(4″-Chlorobenzophenone-2″-formyl)-amino-4′-O-demethylepipodophyllotoxin (Compound No. 8),
4-β-(2″-Chloropyridine-3″-formyl)-amino-4′-4′-O-demethylepipodophyllotoxin (Compound No. 9),
4-β-(6″-Chloropyridine-3″-formyl)amino-4′-O-demethylepipodophyllotoxin (Compound No. 10),
4-β-(Benzene sulphonyl)amino podophyllotoxin (Compound No. 11),
4-β-(p-Toulene sulphonyl)amino podophyllotoxin (Compound No. 12),
4-β-(Benzene sulphonyl)amino-4′-O-demethylepipodophyllotoxin (Compound No. 13),
4-β-(p-Toluene sulphonyl)amino-4′-O-demethylepipodophyllotoxin (Compound No. 14),
4-β-(4″-Chloro-6″-methylpyrimidine-2″-amino)podophyllotoxin (Compound No. 15),
4-β-(Benzothiazole-2″-amino)podophyllotoxin (Compound No. 16),
4-β-(6″-Fluorobenzothiazole-2″-amino)podophyllotoxin (compound No. 17),
4-β-(4″-Chloro-6″-methylpyrimidine-2″-amino)-4′-O-demethylepipodophyllotoxin (Compound No. 18),
4-β-(6″-Chloro-2″-thiomethylpyrimidine-4″-amino)-4′-O-demethylepipodohyllotoxin (Compound No. 19),
4-β-(2″-Chloroacetamido)podophyllotoxin (Compound No. 20),
4-β-[2″-(1,4-Oxazinan-4-yl)acetamido]podophyllotoxin (Compound No. 21),

In the above syntheses, where specific bases, solvents etc., are mentioned, it is to be understood that the other bases, solvents etc., may be used. Similarly, the reaction temperature and duration of the reaction may be adjusted according to the desired need.

The examples mentioned below demonstrate the general synthetic procedure and should not be constrained to limit the scope of the present invention. The compounds were characterized using NMR, IR, MS and were purified by choromatography. Crude products were subjected to column chromatography using silica gel (100-200 or 60-120 mesh) as the stationary phase.

### EXAMPLES

Various solvents, such as methanol, ethanol, chloroform etc. were dried using various drying reagents according to the procedure described in the literature. IR spectra were recorded as nujol mulls or a thin film on a Perkin Elmer Paragon, Nuclear Magnetic Resonance were recorded on a Varian XL-200 instrument using tetramethylsilane as internal standard.

### Example A

### Isolation, Preparation of Podophyllotoxin and Related Compounds

The source of podophyllotoxin was INFAR (India) Ltd., West Bengal, India.

Podophyllotoxin was isolated from the roots and rhizomes of Podophyllum peltatum and Podphyllum emodi as described in Fortschr. Chem. Org. Natursl. 1958, 15, 83. Crude podophyllotoxin (resin enriched with podophyllotoxin) was purified by column chromatography using EtOAc:hexane (3:2) as an eluent followed by recrystallization with CHCl₃/Et₂O.

### Procedure for 4β-amino-4′-O-demethylepipodophyllotoxin

Method 1:

To a solution of crude 4β-azido4′-O-demethylepipodophyllotoxin (2.3 g, 5.4 mmol) in 200 ml of EtOAc was added 500 mg of 10% palladium on carbon. This mixture was shaken under 40 psi of H₂ for 4 h. The reaction mixture was filtered through celite and the filtrate was evaporated in vacuo. This residue was purified by column chromatography (CHCl₃:MeOH, 9:1) to give the pure 4′-demethyl-4β-aminopodophyllotixin (Journal of Natural Products, 1989, 52, 606).

Method 2:

4β-Azidopodophyllotoxin (0.44 g, 1.0 mmol) in MeCN (4 ml) was added to a stirred suspension of sodium iodide (0.36 g, 2.4 mmol) and chlorotrimethy silane (0.5 ml, 2.4 mmol) in MeCN (15 ml) and continued stirring, after the completion of the reaction as indicated by TLC (CHCl₃; MeOH, 9:7: 0.3). The reaction mixture was quenched with saturated sodium thiosulphate solution (15 ml). This reaction mixture was extracted with ethylacetate (30 ml) and washed with brine solution (15 ml). The organic layer was separated and dried over anhydrous Na₂SO₄, and evaporated under vacuum to give the crude product. This was purified by column chromatography (CHCl₃ MeOIl, 9.5:0.5) to give the pure 4β-amino-4-O′-demethylepipodophyllotoxin (Bioorg. Med. Chem. Lett. 1998, 8, 3097).

### Procedure for 4β-aminopodophyllotoxin

Method A

This compound was prepared by employing the above Method 1 (Journal of Natural Products, 1989, 52, 606).

Method B

To a solution of 4β-bromopodophyllotoxin (3 g, 6.2 mmol) in dry dichloromethane (30 ml) was added BaCO₃ (0.6 g, 3mmol) at 0° C. and NH₃ was passed to this solution near to saturation for 20 min and continued stirring at 0° C. till the completion of reaction. BaCO₃ was removed by filtration and the filtrate was extracted with dichloromethane and washed with water. The organic layer was separated, dried over Na₂SO₄ and evaporated under vacuum, to give the crude compound of 4β-aminopodophyllotoxin and this was purified by column chromatography (CHCl₃:MeOH, 9:8; 0.2) to give the pure product in 70% yield (Bioorg. Med. Chem. Lett 1998, 8, 3097).

### Example 1

### Preparation of 4β-(4″-Methylbenzophenone-2″-formyl)aminopodophyllotoxin (Compound No. 1)

Method A: 4β-aminopodophyllotoxin (206 mg, 0.5 mmol) was dissolved in 20 ml of dried dichloromethane, followed by addition of 4-methylbenzophenone-2-carboxylic acid (120 mg, 0.5 mmol) and dicyclohexyl carbodiimide (103 mg, 0.5 mmol). The reaction mixture was stirred at room temperature for about 5 to 10 hours, and then two drops of acetic acid was added. The reaction mixture was filtered and the filtrate was washed with saturated solution of sodium bicarbonate (NaHCO₃), 10% hydrochloric acid and water, dried over anhydrous sodium sulphate (Na₂SO₄) and chromatographed through silica gel using ethyl acetate:hexane (3:7) as eluent to get pure product.

Yield: 174 mg (55%)

Method B: To a solution containing 4β-aminopodophyllotoxin (206 mg, 0.5 mmol) triethylamine (1 ml) in 20 ml of dichloromethane, 4-methylbenzopheonone-2-carboxylic acid chloride (129 mg, 0.5 mmol) in 10 ml of dichloromethane was added under nitrogen and stirred at ambient temperature for about 5 to 10 hours. The reaction mixture was washed with water, extracted with ethyl acetate, dried over anhydrous sodium sulphate (Na₂SO₄) and subjected to column chromatography using ethyl acetate:hexane (3:7) as eluent to afford pure product.

Yield: 269 mg (85%)

m.p: 173-175° C.

[α]_{D}²⁵: −34.5

¹H NMR (CDCl₃): δ 2.9 (s, 3H), 2.8 (m, 2H), 3.8 (s, 3H), 3.9 (s, 6H), 4.1-4.4 (m, 2H), 4.8 (m, 1H), 5.9 (s, 2H), 6.0 (s, 1H), 6.4 (s, 1H), 6.5 (s, 2H), 6.7 (d, 1H, J=6.2 Hz), 7.1 (m, 2H), 7.3 (m, 2H), 7.4 (m, 1H), 7.5 (m, 2H1), 7.7 (d, 2H), 7.8 (d, 1H, J=3.8 Hz).

IR (CHCl₃): 3360, 3290, 2900, 1745, 1690, 1590.

MS: 658 (M⁺ sodium salt), 635 (M⁺), 604, 552, 524, 496, 468, 439, 397, 369, 339.

### Example 2

### Preparation of 4β-(3″-chloro-4″-methylbenzophenone-2″-formyl)amino podophyllotoxin (Compound No. 2)

This compound was prepared according to both the methods described earlier, employing 3-chloro-4-methyl benzophenone-2-carboxylic acid (137 mg, 0.5 mmol), dichlorohexyl carbodiimide (103 mg, 0.5 mmol) and 4β-amino podophyllotoxin (206 mg, 0.5 mmol) to give the product. Data obtained for the process carried out according to Example 1, Method A are given below.

yield: (50%)

m.p: 156-159° C.

[α]_{D}²⁵: −63.5

¹H NMR (CDCl₃): d 2.4 (s, 3H), 2.8 (m, 2H), 3.8 (s, 9H), 3.9-4.2 (m, 2H), 4.5-4.6 (m, 2H), 5.8 (d, 1H, J=5.4 Hz), 5.9 (s, 2H), 6.2 (s, 2H), 6.5 (s, 1H), 6.7 (s, 1H), 7.0 (m, 1H), 7.3 (m, 1H), 7.5 (m, 1H), 7.5 (m, 1H), 7.7 (s, 1H), 7.8 (s, 1H).

IR (CHCl₃): 3300, 3260, 2690, 1725, 1670, 1580.

MS : 671 (M⁺), 669 (M⁺), 578, 551, 397.

### Example 3

### Preparation of 4β-(4″-Chlorobenzophenone-2″-formyl)amino podophyllotoxin (Compound No. 3)

This compound was prepared according to both the methods described earlier, employing 4-chlorobenzophenone-2-carboxylic acid (130 mg, 0.5 mmol), dichlorohexyl carbodiimide (103 mg, 0.5 mmol) and 40-aminopodophyllotoxin (206 mg, 0.5 mmol) to give the product. Data obtained for the process carried out according to Example 1, Method A are given below.

Yield: (75%)

m.p. 143-156° C.

[α]_{D}²⁵: −19.3

¹H NMR (CDCl₃): δ 3.3 (m, 3H), 3.5 (m, 1H), 3.7 (s, 3H), 3.8 (s, 6H), 4.1 (m, 2H), 4.5 (m, 1H), 5.6 (d, 1H), 5.9 (d, 2H, J=3.7 Hz), 6.5 (s, 2H), 7.3 (t, 3H), 7.4 (m, 1H), 7.5 (m, 3H), 7.7 (m, 1H), 7.8 (d, 1H).

IR (CHCl₃): 3300, 3250, 2890, 1750, 1680, 1590.

MS: 655, 603, 577, 551, 523, 412, 397.

### Example 4

### Preparation of 4β-(2″-Chloropyridine-3″-formyl)amino podophyllotoxin (Compound No. 4)

This compound was prepared according to the method A of Example 1 described earlier, employing 2-chloronicotinic acid (78 mg, 0.5 mmol), dichlorohexyl carbodiimide (103 mg, 0.5 mmol) and 4β-aminopodophyllotoxin (206 mg, 0.5 mmol) to give the product.

Yield: (50%)

m.p: 166-168° C.

[α]_{D}²⁵: −136.5

¹H NMR (CDCl₃): δ 2.8-3.0 (m, 2H), 3.7 (s, 9H), 4.0-4.2 (t, 1H), 4.4-4.6 (m, 2H), 5.9 (d, J=5.2 Hz, 2H), 6.3 (s, 1H), 6.5 (s, 2H), (s, 1H), 7.3 (m, 1H), 8.2 (m, 1H), 8.4 (m, 1H).

IR (CHCl₃): 3300, 3290, 2890, 1750, 1570.

MS: 552, 523, 496,467, 397.

### Example 5

### Preparation of 4β-(6″-Chloropyridine-3″-formyl)amino podophyllotoxin (Compound No. 5)

This compound was prepared according to both the methods described earlier, employing 2-chloronicotinic acid (78 mg, 0.5 mmol), dichlorohexyl carbodiimide (103 mg, 0.5 mmol) and 4β-aminopodophyllotoxin (206 mg, 0.5 mmol) to give the product. Data obtained for the process carried out according to Example 1, Method A are given below.

Yield: (45%)

m.p: 173-174° C.

[α]_{D}²⁵: −163.2

¹H NMR (CDCl₃): δ 2.8-3.2 (m, 1H), 3.8 (s, 9H), 4.0-4.2 (m, 2H), 4.4-4.6 (m, 2H), 5.9 (d, J=5.2 Hz, 2H), 6.3 (s, 1H), 6.5 (s, 2H), 6.9 (s, 1H), 7.0 (m, 1H), 7.4 (m, 1H), 8.3 (m, 1H), 8.8 (m, 1H), 8.9 (m, 1H).

IR (CHCl₃): 3300, 3290, 2890, 1750, 1570.

MS: 552, 523, 496, 467, 397.

### Example 6

### Preparation of 4β-(4″-Methylbenzophenone-2″-formyl)amino-4″-O-demethylepipodophyllotoxin (Compound No. 6)

This compound was prepared according to the methods described earlier, employing 4-methylbenzophenone-2-carboxylic acid (120 mg, 0.5 mmol), dichlorohexyl carbodiimide (103 mg, 0.5 mmol) and 4β-amino-4′-O-demethylepipodophyllotoxin (200 mg, 0.5 mmol) to give the product. Data obtained for the process carried out according to Example 1, Method A are given below.

Yield: (85%)

m.p: 184-186° C.

[α]_{D}²⁵: −165.0

¹H NMR (CDCl₃): δ 2.4 (s, 3H), 3.2 (m, 2H), 3.8 (m, 2H), 3.9 (s, 6H), 4.1 (m, 2H), 4.4 (m, 2H), 6.0 (d, J=3.5 Hz, 2H), 6.1 (s, 1H), 6.4 (s, 1H), 6.5 (s, 1H), 6.7 (m, 2H), 7.2 (m, 4H), 7.4 (m, 3H), 7.6 (m, 1H), 7.8 (d, J=5.1 Hz, 1H).

IR (CHCl₃): 3360, 3190, 2920, 1750, 1590.

MS: 622 (M⁺¹), 448, 383, 346, 325.

### Example 7

### Preparation of 4β-(3″-Chloro-4″-Methylbenzophenone-2″-formyl)amino-4′-O-demethylepipodophyllotoxin (Compound No. 7)

This compound was prepared according to both the methods described earlier, employing 3-chloro-4-methylbenzophenone-2-carboxylic acid (137 mg, 0.5 mmol), dichlorohexyl carbodiimide (103 mg, 0.5 mmol) and 4β-amino-4′-O-demethylepipodophyllotoxin (200 mg, 0.5 mmol) to give the product. Data obtained for the process carried out according to Example 1, Method A are given below.

Yield: (80%)

m.p: 177-179° C.

[α]_{D}²⁵: −26.25

¹H NMR (CDCl₃): δ 2.4 (s, 3H), 2.8 (m, 2H), 3.8 (s, 6H), 4.2 (m, 2H), 4.5 (m, 1H), 5.3 (d, 1H), 5.9 (d, 2H, J=2.5 Hz), 6.2 (s, 2H), 6.5 (s, 1H), 6.7 (s, 11), 7.1 (d, 1H), 7.4 (m, 2H), 7.5 (m, 3H), 7.8 (m, 2H), 8.2 (d, 1H, J=4.2 Hz).

IR (CHCl₃): 3300, 3280, 2900, 1745, 1690, 1590.

MS: 656 (M⁺¹), 603, 577, 552, 523, 412, 397.

### Example 8

### Preparation of 4β-(4″-Chlorobenzophenone-2″-formyl)amino-4′-O-demethylepipodophyllotoxin (Compound No. 8)

This compound was prepared according to both the methods described earlier, employing 4-chlorobenzophenone-2-carboxylic acid (130 mg, 0.5 mmol), dichlorohexyl carbodiimide (103 mg, 0.5 mmol) and 4β-amino-4′-O-demethylepipodophyllotoxin (200 mg, 0.5 mmol) to give the product. Data obtained for the process carried out according to Example 1, Method A are given below.

Yield: (85%)

m.p: 182-184° C.

[α]_{D}²⁵: −72.0

¹H NMR (CDCl₃): δ 2.8 (m, 1H), 3.4 (m, 1H), 3.8 (s, 6H), 4.2 (m, 1H), 4.6 (m, 1H), 5.2 (m, 1H), 5.3 (m, 1H), 5.8 (m, 1H), 6.0 (d, 2H, J=2Hz), 6.3 (s, 2H), 6.5 (s, 1H), 6.7 (s, 1H), 7.1 (m, 1H), 7.3 (m, 1H), 7.4 (m, 2H), 7.5 (m, 1H), 7.6 (m, 2H), 7.7 (m, 1H), 7.8 (d, 1H, J=5.2 Hz).

IR (CHCl₃): 3380, 3250, 2900, 1735, 1690, 1580.

MS: 642, 484, 396, 382, 369, 337.

### Example 9

### Preparation of 4β-(2″-Chloropyridine-3″-formyl)amino-4′-O-demethylepipodophyllotoxin (Compound No. 9)

This compound was prepared according to the method A of Example 1 described earlier, employing 2-chloronicotinic acid (78 mg, 0.5 mmol), dichlorohexyl carbodiimide (103 mg, 0.5 mmol) and 4β-amino-4′-O-demethylepipodophyllotoxin (200 mg, 0.5 mmol) to give the product.

Yield: (45%)

m.p: 180-182° C.

[α]_{D}²⁵: −23.5

¹H NMR (CDCl₃): δ 2.9 (m, 1H), 3.1 (m, 1H), 3.8 (s, 6H), 4.0 (m, 1H), 4.5-4.6 (m, 2H), 5.2 (br, 1H), 6.0 (d, 2H, J=7.1 Hz), 6.3 (s, 2H), 6.6 (s, 1H), 6.8 (s, 1H), 7.0 (m, 1H), 7.1 (m, 1H), 8.2 (m, 1H0, 8.5 (d, 1H, J=4.7 Hz).

IR (CHCl₃): 3360, 3280, 2890, 1745, 1580.

MS: 552, 538, 503, 397, 367.

### Example 10

### Preparation of 4β-(6″-Chloropyridine-3″-formyl)amino-4′-O-demethylepipodophyllotoxin (Compound No. 10)

This compound was prepared according to the method A of Example 1 described earlier, employing 6chloronicotinic acid (78 mg, 0.5 mmol), dichlorohexyl carbodiimide (103 mg, 0.5 mmol) and 4β-amino-4′-O-demethylepipodophyllotoxin (200 mg, 0.5 mmol) to give the product.

Yield: (40)

m.p: 185187° C.

[α]_{D}²⁵: −23.5

¹H NMR (CDCl₃): δ 2.9-3.1 (m, 1H), 3.2-3.4 (m, 1H), 3.8 (s, 6H), 3.9-4.0 (m, 2H), 4.3-4.6 (m, 2H), 5.4 (br, 1H), 5.9 (d, 2H, J=5.4 Hz), 6.2 (d, 2H, J=2.7 Hz), 6.8 (s, 1H), 7.1 (m, 1H), 7.4 (d, 1H, J=10.8 Hz), 8.2 (d, 1H, J=16.2 Hz), 8.8 (d, 1H, J=3.2 Hz).

IR (CHCl₃): 3360, 3280, 2910, 1750, 1590.

MS: 540 (M⁺²), 538 (M⁺), 503, 397, 367.

### Example 11

### Preparation of 4β-(Benzenesulphonyl)amino podophyllotoxin (Compound No. 11)

This compound was prepared according to the method B of Example 1 described earlier, employing benzenesulphonyl chloride (0.105 ml, 0.5 mmol), and 4β-aminopodophyllotoxin (206 mg, 0.5 mmol) to give the product.

Yield: (80%)

m.p: 233-235° C.

[α]_{D}²⁵: −76.0

¹H NMR (CDCl₃): δ 2.9 (m, 2H), 3.8 (s, 9H), 4.3-4.4 (m, 1H), 4.5 (m, 2H), 5.6 (s, 1H), 5.9 (d, 2H, J=2.5 Hz), 6.1 (s, 2H), 6.2 (s, 2H), 7.1 (s, 1H), 7.7 (m, 3H), 8.0 (d, 2H, J=10 Hz).

IR (CHCl₃): 3350, 3200, 2860, 1745, 1560.

MS: 553, 397.

### Example 12

### Preparation of 4β-(p-Toluene sulphonyl)aminopodophyllotoxin (Compound No. 12)

This compound was prepared according to the method B of Example 1 described earlier, employing p-toluenesulphonyl chloride (95 mg, 0.5 mmol) and 4β-aminopodophyllotoxin (206 mg, 0.5 mmol) to give the product.

Yield: (85%)

m.p: 209-212° C.

[α]_{D}²⁵: −14.5

¹H NMR (CDCl₃): δ 2.5 (s, 3H), 2.8 (m, 1H), 3.0 (m, 1H), 3.7 (s, 6H), 3.8 (s, 3H), 4.3 (m, 2H), 4.4 (m, 1H), 4.5 (m, 1H), 4.9 (d, 1H, J=5.7 Hz), 5.7 (s, 1H), 5.9 (s, 2H), 6.2 (s, 2H), 6.4 (s, 1H), 7.4 (d, 2H, J=4.5 Hz), 7.8 (d, 2H, J=4.0 Hz)

IR (CHCl₃): 3390, 3280, 2890, 1735, 1580.

MS: 567 (M⁺), 397, 229.

### Example 13

### Preparation of 4β-(Benzene sulphonyl)amino-4′-O-demethylepipodophyllotixin (Compound No. 13)

This compound was prepared according to the method B described earlier, employing benzene sulphonyl chloride (0.105 ml, 0.5 mmol) and 4β-amino-4′-O-demethylepipodophyllotoxin (200 mg, 0.5 mmol) to give the product.

Yield (75%)

m.p: 163-165° C.

[α]_{D}²⁵: −207.50

¹H NMR (CDCl₃): δ 2.8 (m, 1H), 3.2 (m, 1H), 3.7 (s, 6H), 4.0 (m, 2H), 4.4 (m, 1H), 4.7 (m, 1H), 5.9 (d, 2H, J=5.2 Hz), 6.0 (s, 1E), 6.2 (s, 2E), 6.3 (s, 1H), 6.4 (s, 1H), 7.6 (m, 3H), 7.9 (d, 2H, J=4.3 Hz), 8.1 (s, 1H)

IR (CHCl₃): 3380, 3260, 2890, 1735, 1580.

MS: 539, 397.

### Example 14

### Preparation of 4β-(p-Toluene sulphonyl)amino-4′-O-demethylepipodophyllotoxin (Compound No. 14)

This compound was prepared according to the method B of Example 1 described earlier, employing p-toluenesulphonyl chloride (95 mg, 0.5 mmol) and 4β-amino-4′-O-demethylepipodophyllotoxin (200 mg, 0.5 mmol) to give the product.

Yield: (80%)

m.p: 154-156° C.

[α]_{D}²⁵: −55.00

¹HNMR (CDCl₃): δ 2.5 (s, 3H), 2.8-3.0 (m, 2H), 3.8 (s, 6H), 4.3 (m, 2H), 4.4 (m, 2H), 5.6 (s, 1H), 5.9 (d, 2H, J=4.4 Hz), 6.2 (s, 2H), 6.4 (s, 1H), 7.3 (s, 1H), 7.4 (m, 2H), 7.8 (m, 3H), 8.1 (d, 1H, J=3.8 Hz)

IR (CHCl₃): 3400, 3290, 2860, 1750, 1580.

MS: 553, 397.

### Example 15

### Preparation of 4β-(4″-Chloro-6″-methylpyrimidine-2″-amino)podophyllotoxin (Compound No. 15)

To a solution of podophyllotoxin (414 mg, 1 mmol) in dry acetonitrile (10 ml), sodium iodide (298 mg, 2 mmol) was added and stirred for about 5 to 15 min., methanesulphonic acid (192 mg, 2 mmol) was added dropwise at about 0° C. and the stirring was continued for another 0.5 to 1 hours at ambient temperature. Nitrogen was bubbled through the solution to drive of the excess hydrogen iodide. This solution was then evaporated in vacuue and used for the next reaction without further purification. The above crude product, anhydrous barium carbonate (BaCO₃, 395 mg, 2 mmol) and 2-amino-4-chloro-6-methyl pyrimidine (171 mg, 1.2 mmol) in 10 ml of dry tetrahydrofuran under nitrogen was added and stirred for about 8 to 16 hours at ambient temperature. The reaction mixture was dried and purified via column chromatography using ethyl acetate and hexane as eluent to get pure product.

Yield: (80%)

m.p: 198-200° C.

[α]_{D}: −69.00

¹H NMR (CDCl₃): δ 1.3 (s, 3H), 2.7-2.8 (m, 2H), 3.8 (s, 9H), 4.0-4.1 (m, 2H), 4.6-4.8 (m, 2H), 6.0 (d, 2H, J=4.7 Hz), 6.3 (s, 1H), 6.4 (s, 2H), 6.5 (s, 1H), 7.1 (s, 1H).

IR (CHCl₃): 3530-3400, 1750, 1600, 1510.

MS: 541 (M⁺²), 506, 451, 409.

### Example 16

### Preparation of 4β-(3-(Benzothiazole-2″-amino)podophyllotoxin (Compound No. 16)

This compound was prepared according to the method of Example 15 employing 2-aminobenzothiazole (273 mg, 1.2 mmol) and podophyllotoxin (414 mg, 1 mmol) to get pure product.

Yield: (75%)

m.p: 118-121° C.

[α]_{D}: −48.00

¹H NMR (CDCl₃): δ 3.0 (m, 2H), 3.8 (s, 9H), 4.4 (m, 2H), 6.0 (d, 2H, J=4.5 Hz), 6.3 (s, 2H), 6.5 (s, 1), 6.6 (s, 2H), 7.1 (t, 1H), 7.3 (d, 1H, J=6.8 Hz), 7.6 (m, 2H0, 7.9 (s, 1H).

IR (CHCl₃): 3550-3420, 1770, 1610, 1530.

MS: 547 (M⁺¹), 523, 495, 467, 439, 411, 396, 351, 339.

### Example 17

### Preparation of 4β-(6″-Fluorobenzothiazole-2″-amino)podophyllotoxin (Compound No. 17)

This compound was prepared according to the method of Example 15 employing 2-amino-6-fluoro-benzothiazole (201 mg, 1.2 mol) and podophyllotoxin (414 mg, 1 mmol) to get pure product.

Yield: (70%)

m.p: 201-204° C.

[α]_{D}: −48.00

¹H NMR (CDCl₃): δ 3.5 (m, 2H), 3.8 (s, 9H), 4.3 (m, 2H), 4.7-5.2 (m, 2H), (s, 1H), 5.5 9s, 1H), 6.0 (d, 2H, j=5.4 Hz), 6.2 (s, 1H), 6.4 (s, 2H), 6.6 (s, 1H), 7.2-7.5 (m, 3H).

IR (CHCl₃): 3530-3410, 1760, 1600, 1520

MS: 565 (M⁺¹), 552, 537, 523, 509, 467, 451, 439, 428, 411, 395, 383.

### Example 18

### Preparation of 4β-(4″-Chloro-6″-methylpyrimidine-2″-amino)-4′-O-demethylepipodophyllotoxin (Compound No. 18)

To a solution of podophyllotoxin (414 mg, 1 mmol) in dry dichloromethane (10 ml), sodium iodide (447 mg, 3 mmol) was added and stirred for about 5 to 10 min., methanesulphonic acid (288 mg, 3 mmol) was added dropwise at about 0° C. and the stirring was continued for another 5 to 10 hours at ambient temperature. Nitrogen was bubbled through the solution to drive of the excess hydrogen iodide. This solution was then evaporated in vacuue and used for the next reaction without further purification. The above crude product, anhydrous barium carbonate (BaCO₃, 395 mg, 2 mmol) and 2-amino-4-chloro-6-methylpyrimidine (171 mg, 1.2 mmol) in 10 ml of dry tetrahydrofuran under nitrogen were added and stirred for about 8 to hours at ambient temperature. The reaction mixture was filtered, diluted with ethyl acetate and washed with water, 10% sodium thiosulphate solution, dried and purified via column chromatography using ethyl acetate and hexane as eluent to get pure product.

Yield: (50%)

m.p: 205-207° C.

[α]_{D}: −102.0

1H NMR (CDCl₃): δ 2.8 (m, 2H), 3.8 (s, 6H), 3.9 (s, 3H), 4.1 (m, 1H), 4.6 (m, 2H), 4.7 (m, 1H), 5.4 (br, 1H), 6.0 (d, 2H, J=3.4 Hz), 6.4 (s, 2H), 6.5 (s, 1H), 7.1 (s, 1H), 7.3 (d, 1H, J=2.0 Hz).

IR (CHCl₃): 3530-3400, 1750, 1600, 1510.

MS: 527 (M⁺²), 455, 427, 414, 397, 383.

### Example 19

### Preparation of 4β-(6″-Chloro-2″-thiomethylpyrimidine-4″-amino)-4′-O-demethylepipodophyllotoxin (Compound No. 19)

This compound was prepared according to the method of Example 18 employing 4-amino-6-chloro-2-thiomethylpyrimidine (175 mg, 1.2 mmol) and podophyllotoxin (414 mg, 1 mmol) to get pure product.

Yield: (45%)

m.p: 196-199° C.

[α]_{D}: −50.50

¹H NMR (CDCl₃): δ 2.7 (m, 2H), 3.5 (s, 3H), 3.8 (s, 6H), 4.1-4.4 (m, 2H), 4.6-4.8 (m, 2H), 5.3 (br, 1H), 6.0 (d, 2H, J=6.3 Hz), 6.2 (s, 1H), 6.3 (s, 2H), 6.5 (s, 1H), 7.1 (s, 1H).

IR (CHCl₃): 3570-3380, 1720, 1590, 1500.

MS: 559 (M⁺²), 469, 427, 414, 397, 381.

### Example 20

### Preparation of 4β-(2″-Chloroacetamido)podophyllotoxin (Compound No. 20)

To a solution of 4β-amino podophyllotoxin (413 mg, mmol) in dry dichloromethane potassium dicarbonate (280 mg, 2 mmol) was added and stirred for about 5 to 10 minutes, chloroacetylchloride was added dropwise at about 0° C. and the stirring was continued for about 5 to 10 hours at ambient temperature. This solution was evaporated in vaccue and work up with ethyl acetate. The solution was dried subjected to column chromatography using ethyl acetate and hexane as eluent to get the product.

Yield: (80%)

m.p: 145-148° C.

[α]_{D}: −17.00

¹H NMR (CDCl₃): δ 2.8 (dd, 1H), 3.0 (m, 1H), 3.7 (s, 3H), 3.8 (s, 6H), 4.0 (s, 2H), 4.1 (t, 1H), 4.4 (t, 1H), 4.5 (d, 1H), 5.2 (t, 1H), 6.0 (d, 2H), 6.2 (s, 2H), 6.5 (s, 1H), 6.7 (s, 1H), 6.8 (d, 1H).

IR (CHCl₃): 3310, 3240, 1755, 1670, 1590.

MS: 489 (M⁺), 397.

### Example 21

### Preparation of 4β-[2″-(1,4-Oxazinan-4-yl)acetamido]podophyllotoxin (Compound No. 21)

To a solution of the product of Example 20 (489 mg, 1 mmol) in dry acetonitrile potassium dicarbonate (280 mg, 2 mmol) was added and stirred for about 5 to 10 minutes, morpholine (87 mg, 1 mmol) was added at ambient temperature and reflux for about 8 to 15 hours. This solution was evaporated in vacuue and work up with ethyl acetate. The solution was dried and subjected to column chromatography using ethyl acetate and hexane as eluent to get the product.

Yield: (50%)

m.p: 162-164° C.

[α]_{D}: −21.0

¹H NMR (CDCl₃): δ 2.5 (m, 4H), 2.8 (dd, 1EI), 3.0 (m, 1H), 3.4 (m, 4H), 3.7 (s, 6H), 3.8 (s, 3H), 4.4 (t, 1H), 4.6 (d, 1H), 5.2 (t, 1H), 6.0 (s, 2H), 6.2 (s, 1H), 6.5 (s, 1H), 6.7 (s, 1H), 7.1 (d, 1H).

IR (CHCl₃): 3320, 3250, 2870, 1745, 1690, 1570.

MS: 541 (M⁺¹), 397.

Pharmacological Activity

The compounds of the present invention exhibited greater in vitro cytotoxicity values in comparison to etoposide and etoposide resistance cells. Compounds that show activity on these cell lines can be evaluated for in vivo tumor treatment and human tumor xenograft studies. The experiments are designed according to the in vitro screening strategy employed by the National Cancer Institute, USA in their anti-cancer screening program. Therefore, the compounds described herein are provided for the treatment of tumors.

Our screening strategy was based on the screening used by the Developmental Therapeutics Program, National Cancer Institute/National Institute of Health (NCI/NIH), USA. In routine screening, each agent was tested over a broad concentration range (ten-fold dilutions starting from ≧100 μM to ˜10 nM) against 6 human cancer cell lines comprised of different tumor types. Standard compound Doxorubicin was tested in each assay as a positive control. The cells were maintained in growing condition in RPMI 1640 (Rosewell Park Memorial Institute, RPMI-1640 is a commercially available media formulation used for routine culture) medium containing 10% fetal calf serum and incubated at 37° C. under 5% CO₂ atmosphere. All cell lines were inoculated onto a series of standard 96-well microtitre plate on day zero, followed by twenty four hour incubation in the absence of test compound. The inoculation densities used currently in the screen were as per (Monks et al, J. Natl. Cancer Instt., 83, 757 (1991)). All the compounds of the present invention were dissolved in dimethylsulfoxide and diluted further in culture medium. An aliquot of each dilution was added to the growing cells in 96 well plates and incubated for about 48 hrs. After incubation, the assay was terminated by adding 50 μL of Trichloroacetic acid (TCA) and incubating at 4° C. for about 30 min. The precipitated cells were washed and stained with sulphorhodamine B dye for about 30 min and the excess dye was washed off with acetic acid. Adsorbed dye was solubilised in Tris base (alkaline pH) and quantitated by measuring the OD at 490 nm in an Enzyme Linked Immunosorbent Assay (ELISA) reader. GI₅₀ (concentration which inhibits the cell growth by 50%) was calculated according to (Boyd M. R. and Paull K. D. Drug Dev. Res., 34, 91 (1995)).

All compounds tested (Compounds 1-5, 8, 9, 11-13, 16, 17, 20 and 21) exhibited GI₅₀ of less than about 50 μM, with some compounds having GI₅₀ of less than about 10 μM, or les than about 5 μM, or less than about 0.5 μM. Particular compounds showed GI₅₀ of less than about 0.1 μM.

We claim:

1. A compound having the structure of Formula I, 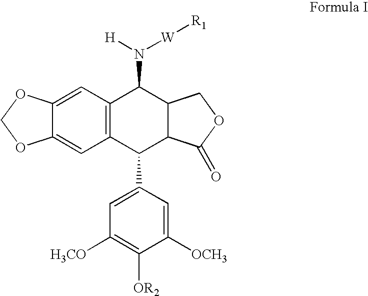
their pharmaceutically acceptable acid addition salts, solvates, enantiomers, diastereomers, metabolites, wherein
R₁ is alkyl, haloalkyl, aryl, heterocyclic, (CH₂)ₙY (wherein Y can represent halogen, amino, nitro or hydroxyl and n can represent an integer 1 to 4), or (CH₂)ₘZ (wherein Z can represent pyridine, piperidine or morpholine and m can represent an integer 1 to 4);
W is no atom, CO, SC, or SO₂; and
R₂ is hydrogen, or alkyl (C₁-C₃).

2. A compound selected from:
4-β-(4″-Methylbenzophenone-2″-formyl)amino podophyllotoxin (Compound No. 1),
4-β-(3″-Chloro-4″-methylbenzophenone-2″-formyl)amino podophyllotoxin (Compound No. 2),
4-β-(4″-Chlorobenzophenone-2″-formyl)amino podophyllotoxin (Compound No. 3),
4-β-(2″-Chloropyridine-3 ″-formyl)amino podophyllotoxin (Compound No. 4),
4-β-(6″-Chloropyridine-3″-formyl)amino podophyllotoxin (Compound No. 5),
4-β-(4″-Methylbenzophenone-2″-formyl)amino-4′-O-demethylepipodophyllotoxin (Compound No. 6),
4-β-(3″-Chloro-4″-methylbenzophenone-2″-formyl)amino-4′-O-demethylepipodophyllotoxin (Compound No.7),
4-β-(4″-Chlorobenzophenone-2″-formyl)-amino-4′-O-demethylepipodophyllotoxin (Compound No. 8),
4-β-(2″-Chloropyridine-3″-formyl)-amino-4′-4′-O-demethylepipodophyllotoxin (Compound No. 9),
4-β-(6″-Chloropyridine-3″-formyl)amino-4′-O-demethylepipodophyllotoxin (Compound No. 10),
4-β-(Benzene sulphonyl)amino podophyllotoxin (Compound No.11),
4-β-(p-Toulene sulphonyl)amino podophyllotoxin (Compound No.12),
4-β-(Benzene sulphonyl)amino-4′-O-demethylpipodophyllotoxin (Compound No. 13),
4-β-(p-Toluene sulphonyl)amino-4′-O-demethylepipodophyllotoxin (Compound No. 14),
4-β-(4″-Chloro-6″-methylpyrimidine-2″-amino)podophyllotoxin (Compound No.15),
4-β-(Benzothiazole-2″-amino)podophyllotoxin (Compound No. 16),
4-β-(6″-Fluorobenzothiazole-2″-amino)podophyllotoxin (Compound No. 17),
4-β-(4″-Chloro-6″-methylpyrimidine-2″-amino)-4′-O-demethylepipodophyllotoxin (Compound No. 18),
4-β-(6″-Chloro-2″-thiomethylpyrimidine-4″-amino)-4′-O-demethylepipodohyllotoxin (Compound No. 19),
4-β-(2″-Chloroacetamido)podophyllotoxin (Compound No.20) and
4-β-[2″-(1,4-Oxazinan-4-yl)acetamido]podophyllotoxin (Compound No.21).

3. A pharmaceutical composition comprising a pharmaceutically effective amount of a compound as defined in claim 1 or 2 together with pharmaceutically acceptable carriers, excipients, or diluents.

4. A method for treating an animal or human suffering from tumor, comprising administering to a patient in need of such treatment a pharmaceutically effective amount of a compound according to claim 1 or 2.

5. A method for treating an animal or human suffering from tumor, comprising administering to a patient in need of such treatment a pharmaceutically effective amount of a pharmaceutical composition according to claim 3.

6. A process for preparing a compound of Formula I, 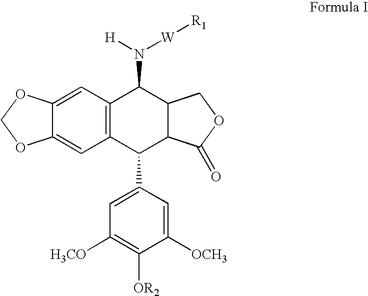
their pharmaceutically acceptable acid addition salts, solvates, enantiomers, diastereomers, metabolites, wherein
R₁ is alkyl, haloalkyl, aryl, heterocyclic, (CH₂)ₙY (wherein Y can represent halogen, amino, nitro or hydroxyl and n can represent an integer 1 to 4), or (CH₂)ₘZ (wherein Z can represent pyridine, piperidine or morpholine and m can represent an integer 1 to 4);
W is no atom, CO, SC, or SO₂; and
R₂ is hydrogen, or alkyl (C₁-C₃),
which method comprises reacting a compound of Formula II with an iodinating agent 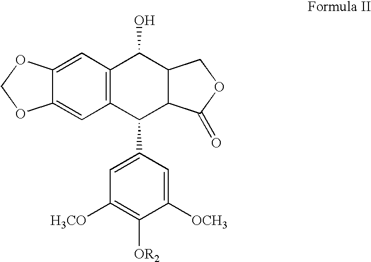
to give a compound of Formula III (wherein R₂ is the same as defined earlier), 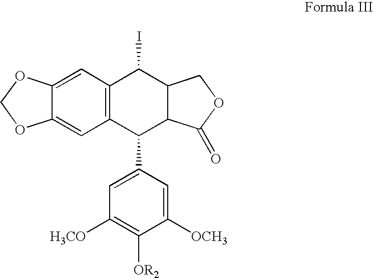
which on reaction with a compound of Formula R₁WNH₂ gives a compound of Formula I (wherein R₁ and W are the same as defined earlier).

7. The process according to claim 6 wherein the reaction of a compound of Formula II to give a compound of Formula III is carried out in a solvent selected from methanol, ethanol, tetrahydrofuran, dimethylformamide and acetonitrile.

8. The process according to claim 6 wherein the reaction of a compound of Formula II to give a compound of Formula III is carried out in acetonitrile.

9. The process according to claim 6 wherein the reaction of a compound of Formula II to give a compound of Formula III is carried out in the presence of an organic acid selected from methanesulfonic acid and p-toluene sulphonic acid.

10. The process according to claim 6 wherein the reaction of a compound of Formula II to give a compound of Formula III is carried out in the presence of methanesulfonic acid.

11. The process according to claim 6 wherein the reaction of a compound of Formula II to give a compound of Formula III is carried out in the presence of an iodinating agent selected from sodium iodide, sodium iodate and potassium dichloroiodate.

12. The process according to claim 6 wherein the reaction of a compound of Formula II to give a compound of Formula III is carried out in the presence of sodium iodide.

13. The process according to claim 6 wherein the reaction of a compound of Formula II to give a compound of Formula III is carried out at temperature ranging from 0° C. to 0° C.

14. The process according to claim 6 wherein the reaction of a compound of Formula II to give a compound of Formula III is carried out at temperature ranging from 0° C. to 5° C.

15. The process according to claim 6 wherein the reaction of a compound of Formula III with a compound of Formula R₁WNH₂ to give a compound of Formula I is carried out in a solvent selected from tetrahydrofuran, dimethylformamide, methanol, ethanol, dichloromethane and acetonitrile.

16. The process according to claim 6 wherein the reaction of a compound of Formula III with a compound of Formula R₁WNH₂ is carried out in acetonitrile.

17. The process according to claim 6 wherein the reaction of a compound of Formula III with a compound of Formula R₁WNH₂ is carried out in the presence of an inorganic base selected from barium carbonate, calcium carbonate, potassium carbonate and sodium bicarbonate.

18. The process according to claim 6 wherein the reaction of a compound of Formula III with a compound of Formula R₁WNH₂ is carried out in the presence of barium carbonate.

19. A process for preparing the compound of Formula I, 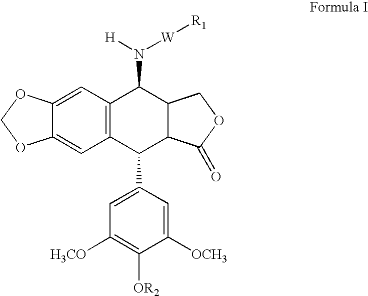
their pharmaceutically acceptable acid addition salts, solvates, enantiomers, diastereomers, metabolites, wherein
R₁ is alkyl, haloalkyl, aryl, heterocyclic, (CH₂)ₙY (wherein Y can represent halogen, amino, nitro or hydroxyl and n can represent an integer 1 to 4), or (CH₂)ₘZ (wherein Z can represent pyridine, piperidine or morpholine and m can represent an integer 1 to 4);
W is no atom, CO, SC, or SO₂; and
R₂ is hydrogen, or alkyl (C₁-C₃).
which method comprises of reacting a compound of Formula IV with a compound of Formula R₁WCOOH 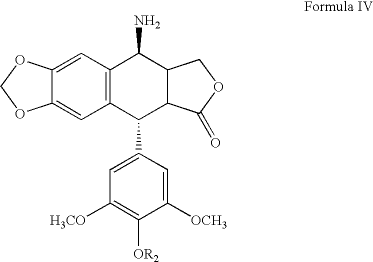
to give a compound of Formula I (wherein R₁, R₂ and W are the same as defined earlier).

20. The process according to claim 19 wherein the reaction of a compound of Formula IV with a compound of Formula R₁WCOOH to give a compound of Formula I is carried out in a solvent selected from dichloromethane, methanol, ethanol, acetonitrile, tetrahydrofuran and dimethylformamide.

21. The process according to claim 19 wherein the reaction of a compound of Formula IV with a compound of Formula R₁WCOOH is carried out in dichloromethane.

22. The process according to claim 19 wherein the reaction of a compound of Formula IV with a compound of Formula R₁WCOOH is carried out in the presence of and activating agent selected from dichlorohexyl carbodiimide and 1-ethyl-3(3-dimethylaminopropyl)carbodiimide.

23. The process according to claim 19 wherein the reaction of a compound of Formula IV with a compound of Formula R₁WCOOH is carried out in the presence of dichlorohexyl carbodiimide.

24. A process for preparing the compounds of Formula I, 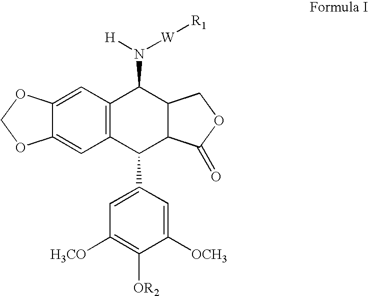
their pharmaceutically acceptable acid addition salts, solvates, enantiomers, diastereomers, metabolites wherein
R₁ is alkyl, haloalkyl, aryl, heterocyclic, (CH₂)ₙY (wherein Y can represent halogen, amino, nitro or hydroxyl and n can represent an integer 1 to 4), or (CH₂)ₘZ (wherein Z can represent pyridine, piperidine or morpholine and m can represent an integer 1 to 4);
W is no atom, CO, SC, or SO₂; and
R₂ is hydrogen, or alkyl (C₁-C₃).
which method comprises of reacting a compound of Formula IV with a compound of Formula R₁WX (wherein X is halogen) 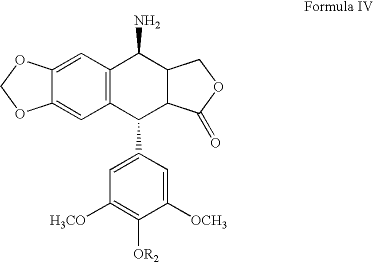
to give a compound of Formula I (wherein R₁, R₂ and W are the same as defined earlier).

25. The process according to claim 24 wherein the reaction of a compound of Formula IV with a compound of Formula R₁WX to give a compound of Formula I is carried out in the presence of a base selected from barium carbonate, calcium carbonate, potassium carbonate, triethylamine and pyridine.

26. The process according to claim 24 wherein the reaction of a compound of Formula IV with a compound of Formula R₁WX is carried out in the presence of triethylamine.
